(19) United States
(12) Patent Application Publication
(10) Pub. No.: US 2023/0310309 A1
(43) Pub. Date: Oct. 5, 2023

(54) INJECTABLE DEPOT COMPOSITIONS FOR THE DELIVERY OF ANTIVIRAL AGENTS

(71) Applicant: Merck Sharp & Dohme LLC, Rahway, NJ (US)

(72) Inventors: Stephanie Elizabeth Barrett, Cranford, NJ (US); Seth P. Forster, Fort Washington, PA (US); Marian E. Gindy, Haledon, NJ (US)

(73) Assignee: Merck Sharp & Dohme LLC, Rahway, NJ (US)

(21) Appl. No.: 18/041,816

(22) PCT Filed: Aug. 23, 2021
(86) PCT No.: PCT/US2021/047046

§ 371 Date: Feb. 16, 2023

Related U.S. Application Data
(60) Provisional application No. 63/069,953, filed on Aug. 25, 2020.

(51) Int. Cl. A61K 9/00; A61K 31/7076; A61K 47/14; A61K 47/20

(52) U.S. Cl. CPC A61K 9/0024; A61K 31/7076; A61K 47/14; A61K 47/20

(57) ABSTRACT

This invention relates to novel injectable depot compositions for long-acting delivery of antiviral drugs. These compositions are useful for the treatment or prevention of human immunodeficiency virus (HIV) infection.

### BACKGROUND OF THE INVENTION

The development of highly active antiretroviral therapy (HAART) in the mid 1990's transformed the clinical care of human immunodeficiency virus (HIV) type infection. HAART regimens have proven to be highly effective treatments, significantly decreasing HIV viral load in HIV-infected patients, thereby slowing the evolution of the illness and reducing HIV-related morbidity and mortality. Yet, the treatment success of HAART is directly related to adherence to the regimen by the patient. Unless appropriate levels of the antiretroviral drug combinations are maintained in the blood, viral mutations will develop, leading to therapy resistance and cross-resistances to molecules of the same therapeutic class, thus placing the long-term efficacy of treatments at risk. Various clinical studies have shown a decline in treatment effectiveness with relatively small lapses in adherence. A study by Musiime found that 81% of patients with more than 95% adherence demonstrated viral suppression, while only 50% of patients who were 80-90% adherent were successful. See, Musiime, S., et al., Adherence to Highly Active Antiretroviral Treatment in HIV-Infected Rwandan Women. PLOS one 2011, 6, (11), 1-6. Remarkably, only 6% of patients that were less than 70% adherent showed improvements in viral markers. Thus, low adherence is a leading cause of therapeutic failure in treatment of HIV-1 infection.

Nonetheless, adherence rates to the HAART regimens continue to be far from optimal. Various characteristics of HAART make adherence particularly difficult. Therapeutic regimens are complex, requiring multiple drugs to be taken daily, often at different times of the day, and many with strict requirements on food intake. Many HAART medications also have unpleasant side effects, including nausea, diarrhea, headache, and peripheral neuropathy. Social and psychological factors can also negatively impact adherence. Patients report that forgetfulness, lifestyle factors, including fear of being identified as HIV-positive, and therapy fatigue over life-long duration of treatment all contribute to adherence lapses.

New HIV treatment interventions aim to improve adherence by reducing the complexity of treatments, the frequency of the dosages, and/or the side effects of the medications. Long-acting injectable (LAI) drug formulations that permit less frequent dosing, on the order of a month or longer, are an increasingly attractive option to address adherence challenges. However, the majority of approved and investigational antiretroviral agents are not well suited for reformulation as long-acting injectable products. In large part, this is due to suboptimal physicochemical properties limiting their formulation as conventional drug suspensions, as well as insufficient antiviral potency resulting in high monthly dosing requirements. Even for cabotegravir or rilpivirine, two drugs being studied as long-acting injectable formulations, large injection volumes and multiple injections are required to achieve pharmacokinetic profiles supportive of monthly dosing. See, e.g., Spreen, W. R., et al., Long-acting injectable antiretrovirals for HIV treatment and prevention. Current Opinion in Hiv and Aids 2013, 8, (6), 565-571; Rajoli, R. K. R., et al., Physiologically Based Pharmacokinetic Modelling to Inform Development of Intramuscular Long-Acting Nanoformulations for HIV. Clinical Pharmacokinetics 2015, 54, (6), 639-650; Baert, L., et al., Development of a long-acting injectable formulation with nanoparticles of rilpivirine (TMC278) for HIV treatment. European Journal of Pharmaceutics and Biopharmaceutics 2009, 72, (3), 502-508; Van't Klooster, G., et al., Pharmacokinetics and Disposition of Rilpivirine (TMC278) Nanosuspension as a Long-Acting Injectable Antiretroviral Formulation. Antimicrobial Agents and Chemotherapy 2010, 54, (5), 2042-2050. Thus, novel formulation approaches capable of delivering extended-duration pharmacokinetic characteristics for antiviral agents at practical injection volumes and with a limited number of injections are highly desirable.

### SUMMARY OF THE INVENTION

This invention relates to novel injectable depot compositions for long-acting delivery of antiviral drugs. These compositions are useful for the treatment or prevention of human immunodeficiency virus (HIV) infection.

### BRIEF DESCRIPTION OF THE DRAWINGS

FIG. 1 shows in vitro islatravir release rate from the injectable depot compositions when varying the drug loading of islatravir.

FIG. 2 shows in vitro islatravir release rate from the injectable depot compositions when varying the polymer.

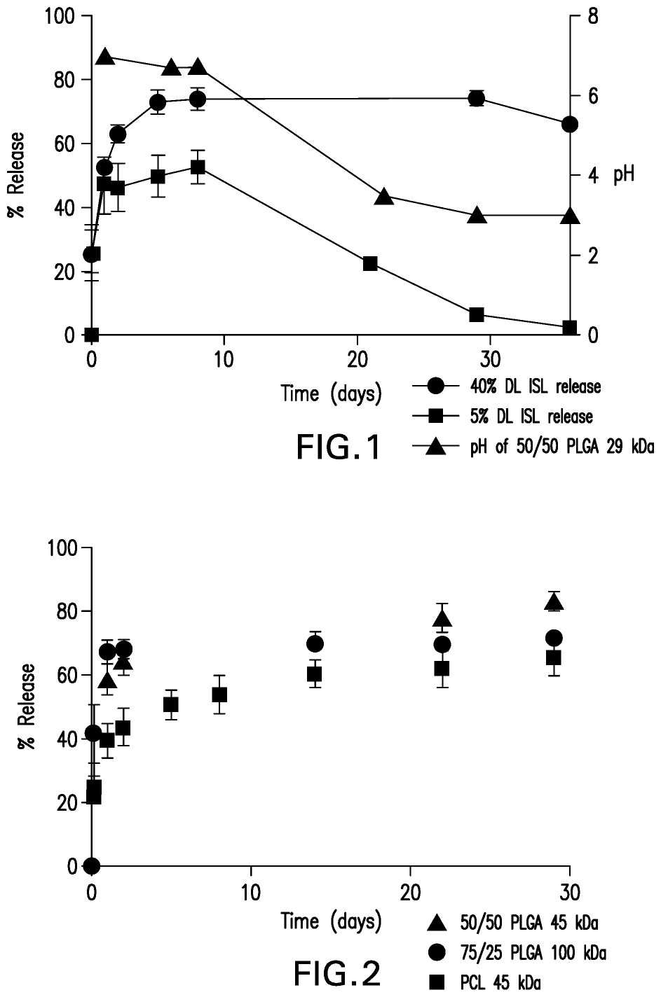

### DETAILED DESCRIPTION OF THE INVENTION

This invention relates to novel injectable depot compositions for long-acting delivery of antiviral drugs. The novel injectable depot compositions of the instant invention comprise a polymer, an antiviral agent and a solvent. These injectable depot compositions are useful for the treatment or prevention of human immunodeficiency virus (HIV) infection. The invention further relates to methods of treating and preventing HIV infection with the novel injectable depot compositions described herein.

The novel injectable depot compositions of the instant invention comprise 10% to 50% of a biocompatible bioerodible polymer by weight, 3% to 40% of islatravir, or a pharmaceutically acceptable salt thereof, by weight and 30% to 85% of a solvent by weight. The biocompatible bioerodible polymer is selected from the group consisting of poly(DL-lactide), poly(caprolactone), poly(lactic-co-glycolic acid), poly(lactide), poly(glycolide), poly(ε-caprolactone), poly(ortho esters), poly(dioxanone) and combinations thereof. In an embodiment of the invention, the biocompatible bioerodible polymer is poly(lactic-co-glycolic acid). The solvent is selected from the group consisting of ethyl benzoate, N-methyl-2-Pyrrolidone, dimethylsulfoxide, benzyl benzoate, benzyl alcohol, poly(ethylene glycol) dimethyl ether, triacetin, glycofurol and mixtures thereof. In an embodiment of the invention, the solvent is dimethylsulfoxide.

In the injectable depot compositions of the instant invention, the islatravir, or a pharmaceutically acceptable salt thereof, is present in the composition between 8% to 20% by weight. The biocompatible bioerodible polymer is present in the composition between 15% to 30% by weight. The solvent is present in the composition between 55% to 75% by weight. In an embodiment of the invention, the islatravir, or a pharmaceutically acceptable salt thereof, is present in the composition between 8% to 18% by weight, the polymer is present in the composition between 15% to 27% by weight and the solvent is present in the composition between 55% to 75% by weight.

In an embodiment of the injectable depot compositions of the instant invention, the ratio of the lactic acid to glycolic acid of the poly(lactic-co-glycolic acid) is 75:25. In a class of the embodiment, the weight of the 75:25 poly(lactic-co-glycolic acid) is 100 kDa. In another embodiment of the injectable depot compositions of the instant invention, the ratio of the lactic acid to glycolic acid of the poly(lactic-co-glycolic acid) is 50:50. In a class of the embodiment, the weight of the 50:50 poly(lactic-co-glycolic acid) is 29 kDa.

An injectable depot composition of the present invention comprises 18% of islatravir by weight, 27% of 75:25 poly(lactic-co-glycolic acid) 100 kDa by weight and 55% dimethylsulfoxide by weight. Another injectable depot composition of the present invention comprises 18% of islatravir by weight, 27% of 50:50 poly(lactic-co-glycolic acid) 29 kDa by weight and 55% dimethylsulfoxide by weight.

The injectable depot compositions of the present invention form a biodegradable implant in situ and are injected into a patient in need thereof subdermally. The compositions are useful for treating or preventing HIV infection, and the islatravir is released at therapeutic concentrations of one month or between three and six months.

The novel injectable depot compositions of the invention comprise a biocompatible bioerodible polymer with a drug and solvent. The chemical properties of the polymer matrices are tuned to achieve a range of drug release characteristics, offering the opportunity to extend duration of dosing. In an embodiment of the invention, the novel injectable depot compositions are compatible with molecules having a broad spectrum of physicochemical properties, including those of high aqueous solubility or amorphous phases which are unsuitable to formulation as solid drug suspensions.

Specifically, this invention relates to novel injectable depot compositions comprising islatravir, a biocompatible bioerodible polymer and a solvent. These injectable depot compositions can be injected subcutaneously or intramuscularly wherein islatravir is continually released in vivo at a rate resulting in a plasma concentration between 0.01 ng/mL and 10,000 ng/mL. These injectable depot compositions are desired and useful for prophylaxis and/or treatment of HIV infection from both compliance and convenience standpoints.

As used herein, the polymeric materials for use in the invention contain enzymatically or hydrolytically labile linkages which undergo cleavage at physiological conditions. The polymeric materials must be both “biocompatible” and “bioerodible.” Biocompatible polymers, in a broad sense, have properties that make them compatible with the tissues of the subject into which they will be implanted. These polymers are suitable for implanting in vivo and have acceptable safety and tolerability profiles. Bioerodible polymers are those that gradually decompose, dissolve, hydrolyze and/or erode in situ (in the body). The broken down products are generally non-toxic and are excreted. The polymer is generally hydrophobic so that it retains its integrity for a suitable period of time when placed in an aqueous environment. Bioerodible polymers remain intact in vivo for extended periods of time, typically weeks, months or years. Drug molecules encapsulated in the polymer are released over time via diffusion through channels and pores in a sustained manner. The release rate can be altered by modifying the identity of the polymer (for example, monomeric units, molecular weight, end group, etc.) thereby modifying the degradation kinetics of the polymer, porosity of the polymer or hydrophobicity of the polymer.

Accordingly, a polymer that can be readily cleared or eliminated by the body can be used to manufacture the injectable depot compositions of the instant invention. Biocompatible bioerodible polymers of the instant invention include, but are not limited to, poly(DL-lactide) (“PLA”), poly(caprolactone) (“PCL”), poly(lactide-co-glycolic acid) (“PLGA”), poly(lactide), poly(glycolide), poly(ε-caprolactone), poly(ortho esters), poly(amines), poly(urethanes), poly(amino acids), poly(malic acid), poly(ketals), poly(acetals), poly(anhydrides), poly(ester amides), poly(dioxanone), poly(saccharides), poly(ethylene glycol), chitin, chitosan and combinations thereof. In a class of the invention, the biocompatible bioerodible polymer is poly(lactide-co-glycolic acid).

In one embodiment, the biocompatible bioerodible polymer of the invention is poly(lactic acid-co-glycolic acid), which is a copolymer based on lactic acid and glycolic acid. The polymer can include small amounts of other comonomers that do not substantially affect the advantageous results that can be achieved in accordance with the invention. The term “lactic acid” includes the isomers L-lactic acid, D-lactic acid, DL-lactic acid, and lactide. The term “glycolic acid” includes glycolide. The polymer may have a lactic acid to glycolic acid monomer ratio of from 100:0 to 15:85. In an embodiment of the invention, the lactic acid to glycolic acid monomer ratio is from 60:40 to 75:25. In another embodiment of the invention, the lactic acid to glycolic acid monomer ratio is 50:50. The polylactide polymer has an average molecular weight ranging from about 1,000 to about 120,000, as determined by gel permeation chromatography. In an embodiment of the invention, the polylactide polymer has a number average molecular weight ranging from about 30,000 to about 100,000, as determined by gel permeation chromatography. Suitable polylactide polymers are available commercially.

In an embodiment of the invention, the biocompatible bioerodible polymer is poly(lactic-co-glycolic acid). In a class of the invention, the ratio of the lactic acid to glycolic acid of the poly(lactic-co-glycolic acid) is 75:25. In a subclass of the invention, the weight of the 75:25 poly(lactic-co-glycolic acid) is 100 kDa. In another class of the invention, the ratio of the lactic acid to glycolic acid of the poly(lactic-co-glycolic acid) is 50:50. In a subclass of the invention, the weight of the 50:50 poly(lactic-co-glycolic acid) is 29kDa.

The injectable depot compositions further include a biocompatible solvent which, when combined with the polymer, forms a viscous gel typically exhibiting viscosity in a range from 500 poise to 200,000 poise. In an embodiment of the invention, the viscosity is from 1,000 poise to 50,000 poise. The solvent used in the injectable depot composition is typically an organic solvent and may be a single solvent or a mixture of solvents. To limit water intake by the injectable depot compositions in the environment of use, the solvent, or at least one of the components of the solvent in the case of a multi-component solvent, has limited miscibility with water. In an embodiment of the invention, the solvent has less than 7% by weight miscibility with water. In a class of the invention, the solvent has less than 5% by weight miscibility with water. In a subclass of the invention, the solvent has less than 3% miscibility by weight with water. Examples of suitable solvents include, but are not limited to, ethyl benzoate (EB), N-methyl-2-pyrrolidone (NMP), dimethylsulfoxide (DMSO), benzyl benzoate (BB), benzyl alcohol (BA), poly(ethylene glycol) dimethyl ether, triacetin, glycofurol and mixtures thereof. In an embodiment of the invention, the solvent is dimethylsufloxide.

As used herein, the term “continually released” refers to the drug being released from the injectable depot compositions at continuous rates for extended periods of time.

The novel injectable depot compositions of the invention comprise antiviral agents. Suitable antiviral agents include anti-HIV agents. In an embodiment of the invention, the antiviral agent is administered as a monotherapy. In another embodiment of the invention, two or more antiviral agents are administered in combination.

An “anti-HIV agent” is any agent which is directly or indirectly effective in the inhibition of HIV reverse transcriptase or another enzyme required for HIV replication or infection, the or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. Suitable anti-viral agents for use in implant drug delivery systems described herein include, for example, those listed in Table A as follows:

TABLE A
Antiviral Agents for Preventing HIV infection or AIDS

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| Capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, Atripla ® | nnRTI + nRTI |
| islatravir | nRTI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| Lenacapavir | CI |
| Lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| PPL-100 (also known as PL-462) (Ambrilia) | PI |
| raltegravir, Isentress ™ | InI |
| (S)-2-(3-chloro-4-fluorobenzyl)-8-ethyl-10-hydroxy- | InI |
| N,6-dimethyl-1,9-dioxo-1,2,6,7,8,9- | |
| hexahydropyrazino[1′,2′:1,5]pyrrolo[2,3-d]pyridazine- | |
| 4-carboxamide (MK-2048) | |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| Tenofovir, hexadecyloxypropyl (CMX-157) | nRTI |
| tipranavir, Aptivus ® | PI |
| Vicriviroc | EI |

EI=entry inhibitor; FI=fusion inhibitor; InI=integrase inhibitor; PI=protease inhibitor; nRTI=nucleoside reverse transcriptase inhibitor; nnRTI=non-nucleoside reverse transcriptase inhibitor; CI=capsid inhibitor.

Some of the drugs listed in the table can be used in a salt form; e.g., abacavir sulfate, delavirdine mesylate, indinavir sulfate, atazanavir sulfate, nelfinavir mesylate, saquinavir mesylate.

In certain embodiments the antiviral agents in the injectable depot compositions described herein are employed in their conventional dosage ranges and regimens as reported in the art, including, for example, the dosages described in editions of the Physicians' Desk Reference, such as the 70th edition (2016) and earlier editions. In other embodiments, the antiviral agents in the implant drug delivery systems described herein are employed in lower than their conventional dosage ranges.

In an embodiment of the invention, the antiviral agent can be an entry inhibitor; fusion inhibitor; integrase inhibitor; protease inhibitor; nucleoside reverse transcriptase inhibitor; or non-nucleoside reverse transcriptase inhibitor. In a class of the invention, the antiviral agent is a nucleoside reverse transcription inhibitor.

In an embodiment of the invention, the antiviral agent is a nucleoside reverse transcriptase inhibitor (NRTI). In a class of the invention, the NRTI is islatravir.

Islatravir is also known as 4′-ethynyl-2-fluoro-2′-deoxyadenosine and EFdA, and has the following chemical structure:

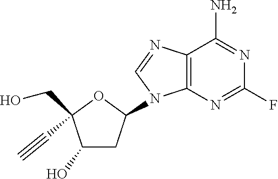 [SMILES: C#C[C@]1(CO)O[C@@H](n2cnc3c(N)nc(F)nc32)C[C@@H]1O]

Production of and the ability of islatravir to inhibit HIV reverse transcriptase are described in PCT International Application WO2005090349, published on Sep. 29, 2005, and US Patent Application Publication No. 2005/0215512, published on Sep. 29, 2005, both to Yamasa Corporation which are hereby incorporated by reference in their entirety.

Small molecule drug formulations according to embodiments of the invention can be prepared as injectable depot compositions. The environment of use is a fluid environment and as such the compositions can be injected into a subcutaneous, intramuscular, intranodal or lymphatic space of a human or animal. Multiple or repeated injections may be administered to the subject, for example, when the therapeutic effect of the drug has subsided or the period of time for the drug to have a therapeutic effect has lapsed or when the subject requires further administration of the drug for any reason. The injectable depot composition forms a biodegradable implant in situ. The injectable depot composition releases the antiviral or antivrials in a sustained manner over a period of one week, more than one week, one month, or more than one month. In an embodiment of the invention, the release of the antiviral or antivirals is over at least a period of one month. In another embodiment of the invention, the release of the antiviral or antivirals is over a period of at least 1 to 3 months. In another embodiment of the invention, the release of the antiviral or antivirals is over a period of 3 to 6 months.

In an embodiment of the injectable depot compositions described herein, the islatravir, or pharmaceutically acceptable salt thereof, is present in composition between 3% to 40% by weight. In a class of the embodiment, the islatavir is present in the composition between 8% to 20% by weight. In another class of the embodiment, the islatavir is present in the composition between 8% to 18% by weight. In a subclass of the embodiment, islatravir is present in the composition at 8% by weight. In another subclass of the embodiment, islatravir is present in the composition at 10% by weight. In another subclass of the embodiment, islatravir is present in the composition at 11% by weight. In another subclass of the embodiment, islatravir is present in the composition at 12% by weight. In another subclass of the embodiment, islatravir is present in the composition at 14% by weight. In another subclass of the embodiment, islatravir is present in the composition at 18% by weight.

In an embodiment of the injectable depot compositions described herein, the biocompatible bioerodible polymer is present in composition between 10% to 50% by weight. In a class of the embodiment, the biocompatible bioerodible polymer is present in the composition between 15% to 30% by weight. In another class of the embodiment, the biocompatible bioerodible polymer is present in the composition between 15% to 27% by weight. In a subclass of the embodiment, biocompatible bioerodible polymer is present in the composition at 15% by weight. In another subclass of the embodiment, biocompatible bioerodible polymer is present in the composition at 17% by weight. In another subclass of the embodiment, biocompatible bioerodible polymer is present in the composition at 18% by weight. In another subclass of the embodiment, biocompatible bioerodible polymer is present in the composition at 27% by weight.

In an embodiment of the injectable depot compositions described herein, the solvent is present in composition between 30% to 85% by weight. In a class of the embodiment, the solvent is present in the composition between 55% to 75% by weight. In a subclass of the embodiment, the solvent is present in the composition at 55% by weight. In another subclass of the embodiment, the solvent is present in the composition at 75% by weight.

The injectable depot compositions described herein are capable of releasing islatravir over a period of 21 days, 28 days, 31 days, 4 weeks, 6 weeks, 8 weeks, 12 weeks, one month, two months, three months, four months, five months, six months, seven months, eight months, nine months, ten months, eleven months, twelve months, eighteen months, twenty-four months or thirty-six months at an average rate of between 0.01-5 mg per day. In an embodiment of the invention, the islatravir is released at therapeutic concentrations for a duration from between one month and three months. In a class of the embodiment, the islatravir is released at therapeutic concentrations for a duration from between three months and six months.

The injectable depot compositions described herein are capable of islatravir releasing resulting in a plasma concentration of between 0.01-100 ng/mL per day. In an embodiment of the invention, the implant drug delivery systems described herein are capable of releasing islatravir resulting in a plasma concentration of between 0.1-5.0 ng/mL per day. In a class of the embodiment, the implant drug delivery systems described herein are capable of releasing islatravir resulting in a plasma concentration of between 0.1-2 ng/mL per day. In a subclass of the embodiment, the implant drug delivery systems described herein are capable of releasing islatravir resulting in a plasma concentration of between 0.1-1.0 ng/mL per day.

The following examples are given for the purpose of illustrating the present invention and shall not be construed as being limitations on the scope of the invention.

### EXAMPLE 1

### Preparation of Compositions Containing Islatravir

The formulation requires that islatravir and the bioerodible polymer are dissolved in solvent. In this example, islatravir and a bioerodible polymer, PLGA, are dissolved in solvent at a temperature of 50° C. for 72 hours. In this example, the weight % ratio of the PLGA, islatravir, and solvent are varied in the compositions. The compositions maintained a low viscosity, which is acceptable for administration by injection to a subject.

Injectable depot compositions were prepared according to the following procedure:

The polymer (75/25 lactic acid: glycolic acid, 75/25 PLGA 100 kDa, Sigma Aldrich, Product #: 719927)) and drug (islatravir) were added to organic solvent (dimethylsulfoxide, product ID:41639 lot BCBH5215V from Sigma Aldrich) and allowed to dissolve for 72 h at 50° C. on an Eppendorf mixer shaking at 700 RPM. Polymer, drug and solvent were added at different wt % for the various compositions as noted in Table 1.

TABLE 1
Compositions of injectable depot compositions of islatravir (ISL)

| Mass | | Mass | | Mass | |
|---|---|---|---|---|---|
| ISL | wt % | PLGA | wt % | DMSO | wt % |
| (mg) | ISL | (mg) | PLGA | (mg) | solvent |
| 404 | 27 | 267 | 18 | 809 | 55 |
| 402 | 14 | 613 | 21 | 1858 | 65 |
| 402 | 6 | 1607 | 23 | 4850 | 71 |
| 400 | 3 | 3602 | 24 | 10921 | 73 |

Note:
PLGA = 75/25 lactic acid:glycolic acid.

### EXAMPLE 2

### In Vitro Release of Islatravir From Compositions With Variable Solvents

The preparation of the formulations for this example follows the methodology described in Example 1. This example investigates influence of solvent type on in vitro release of islatravir from depot gel compositions. For each solvent system studied, the formulation was optimized so that the highest percentage solids loading was achieved. The findings suggest specific solvent systems allowed for higher drug solubilization while maintaining low viscosity acceptable for administration by injection to a subject.

Injectable depot compositions of islatravir, 50/50 lactic acid:glycolic acid PLGA 28.5 kDa and various solvents (see Table 3 were prepared according to the following procedure:

The polymer (50/50 PLGA 28.5 kDa, Sigma Aldrich, Product #: 719870)) and drug (islatravir) were dissolved into organic solvent (see specific solvent in table) and allowed to dissolve for 72 h at 50° C. on an Eppendorf mixer shaking at 700 RPM. Depot compositions were prepared by adding the organic solution of islatravir and polymer to phosphate buffered saline (PBS). The mass of all components was recorded (mass of vial with PBS, then mass with PBS after the organic solution was added). The injectable depot compositions, which can also be characterized as gels at this stage, were prepared in glass vials. Once the depot gel was formed, it was placed in the oven on an orbital table (with gentle agitation).

TABLE 2
Injectable depot compositions tested

| | | | | | | | | Drug/ | |
|---|---|---|---|---|---|---|---|---|---|
| | | | | | | | | solvent | |
| Mass | | Mass | | Mass | Mass | | | mass added | Volume |
| ISL | wt % | PLGA | wt % | solvent 1 | solvent 2 | Solvent 1/ | wt % | to PBS | PBS |
| (mg) | ISL | (mg) | PLGA | (mg) | (mg) | Solvent 2 | solvent | (mg) | (mL) |
| 328 | 14 | 504 | 22 | 1506 | | NMP | 64 | 282 | 30 |
| 322 | 11 | 496 | 17 | 1007 | 1011 | ethyl | 71 | 293 | 30 |
| | | | | | | benzoate/ | | | |
| | | | | | | DMSO | | | |
| 329 | 8 | 510 | 12 | 2518 | 1002 | benzyl | 81 | 389 | 30 |
| | | | | | | benzoate/ | | | |
| | | | | | | DMSO | | | |
| 340 | 18 | 496 | 26 | 536 | 500 | PEG 500 | 55 | 582 | 30 |
| | | | | | | DME/ | | | |
| | | | | | | DMSO | | | |
| 331 | 18 | 508 | 27 | 1021 | | DMSO | 55 | 229 | 30 |
| 333 | 10 | 506 | 15 | 2527 | | glycofurol | 75 | 429 | 30 |
| 334 | 12 | 503 | 18 | 1015 | 1009 | benzyl | 71 | 323 | 30 |
| | | | | | | alcohol/ | | | |
| | | | | | | DMSO | | | |

Note:
DMSO = dimethylsulfoxide;
PLGA = 50/50 lactic acid:glycolic acid;
PEG 500 DME = poly(ethylene glycol) dimethyl ether

The in vitro release rate of islatravir was determined by placing a specified mass of the drug/solvent into a glass vial containing phosphate buffered saline (PBS). The volume of drug/solvent added was calculated such that when 100% of the drug was released, the drug concentration is at sink conditions, defined here as 1× solubility (the solubility value for islatravir in PBS at 37° C. is 1.35 mg/mL). The resulting gel, submerged in dissolution media, was then added to an Innova 42 incubator shaker, set to 40° C. and orbiting at 50 RPM. Samples were removed at selected time points to assess the percentage islatravir released (by HPLC) over time. To generate in vitro release profiles of the various implants tested, 500 uL of dissolution media was removed from the sample vial at designated time points and centrifuged at 20,800 xg for 8 min. The supernatant was removed (400 uL), samples were diluted 4-fold, and vortexed. Samples were assayed by HPLC (Agilent 1100 series). Analysis of a 6 uL volume was performed at 240 nm with a Supelco Ascentis® Express C18 column (100'4.6 mm, 2.7um). The mobile phase was 0.1% H₃PO₄ and 50:50 ACN (acetonitrile):MeOH (methanol) (83:17 v/v) at a flow rate of 1.5 mL/min (40° C.).

To determine degradation of islatravir by HPLC, a 6 uL volume was injected onto an Agilent Zorbax SB-Aq column (150×4.6 mm, 3.5um). The mobile phase was 0.1% H₃PO₄ and 50:50 ACN:MeOH with a flow rate of 1.0 mL/min (40° C.). The mobile phase gradient is shown in the table below.

TABLE 3
Islatravir chemical stability HPLC method details

| Time (min) | 0.1% H₃PO₄ |
|---|---|
| 0.0 | 98 |
| 10.0 | 95 |
| 12.0 | 90 |
| 14.0 | 10 |
| 14.1 | 98 |
| 20.0 | 98 |

All samples were calibrated to 0.5 mg/mL standard solutions of isltravir in 50:50 MeOH:H₂O.

TABLE 4
The effect of polymer loading and solvent on in vitro release of islatravir
in 50/50 PLGA 28.5 kDa (Avg = average cumulative release, %)

| | | | 17 wt % | | 12 wt % | | 26 wt % | | | | | | 18 wt % | |
|---|---|---|---|---|---|---|---|---|---|---|---|---|---|---|
| | | | polymer in | | polymer in | | polymer in | | | | | | polymer in | |
| | 22 wt % | | EB/DMSO | | BB/DMSO | | PEG500/DMSO | | 27 wt % | | 15 wt % | | BA/DMSO | |
| | polymer in | | 1:1 | | 2.5:1 | | 1:1 | | polymer in | | polymer in | | 1:1 | |
| | NMP | | (v/v %) | | (v/v %) | | (v/v %) | | DMSO | | glycofurol | | (v/v %) | |
| Time | Avg | Std. | Avg | Std. | Avg | Std. | Avg | Std. | Avg | Std. | Avg | Std. | Avg | Std. |
| (days) | (%) | Dev. | (%) | Dev. | (%) | Dev. | (%) | Dev. | (%) | Dev. | (%) | Dev. | (%) | Dev. |
| 0 | 0 | 0 | 0 | 0 | 0 | 0 | 0 | 0 | 0 | 0 | 0 | 0 | 0 | 0 |
| 0.2 | 16 | 4 | 12 | 3 | 9 | 0 | 20 | 0 | 16 | 0 | 42 | 15 | 42 | 15 |
| 1 | 64 | 11 | 48 | 0 | 25 | 0 | 35 | 0 | 25 | 0 | 66 | 19 | 66 | 19 |
| 2 | 78 | 8 | 64 | 4 | 36 | 3 | 39 | 1 | 25 | 0 | 81 | 6 | 81 | 6 |
| 4 | 87 | 4 | 78 | 2 | 58 | 8 | 41 | 1 | 25 | 0 | 88 | 1 | 88 | 1 |
| 7 | 91 | 1 | 88 | 1 | 85 | 7 | 40 | 1 | 79 | 0 | 89 | 1 | 89 | 1 |
| 20 | 90 | 1 | 91 | 1 | 93 | 0 | 58 | 1 | | | 90 | | | |

Note:
EB = ethyl benzoate; NMP = N-methyl-2-Pyrrolidone; DMSO = dimethylsulfoxide; BB = benzyl benzoate; BA = benzyl alcohol; PEG 500 DME = Poly(ethylene glycol) dimethyl ether.
Note:
percentages given in this table reflect the fraction released relative to the total drug in the depot.

### EXAMPLE 3

### Preparation of Compositions Containing Islatravir With Variable Polymer, Polymer Loading and Solvent

The preparation of the formulations for this example follows the methodology in Example 1, and the in vitro drug release methods follow the description from Example 2. This example investigates influence of polymer type on in vivo release of islatravir from injectable depot compositions. The formulations in this example were optimized for release rate of islatravir to provide in vivo durations of up to 4 months.

Injectable depot compositions were prepared as described in Example 1 and tested in vivo in rats to determine release of islatravir as determined by blood serum or plasma concentration of islatravir as a function of time. All animal studies were conducted following protocols in accordance with the Institutional Animal Care and Use Committee (IACUC) at NIRC and Merck & Co., Inc., Kenilworth, New Jersey, U.S.A. which adhere to the regulations outlined in the USDA Animal Welfare Act. Wistar Han rats were anesthetized using isoflurane to effect prior to subcutaneous dose administrations. Animals were dosed at 0.3 mL/kg in the scapular region. Four animals (4 males) were used for each formulation. Animals were monitored until recovered. At indicated time points, samples of blood were obtained from anesthetized animals (using isoflurane) and processed to plasma for determination of 4′-ethynyl-2-fluoro-2′-deoxyadenosine levels.

The injectable depot compositions used in this study were prepared according to the following procedure:

Composition 1: (18% Islatravir (ISL); 27% poly(lactic-co-glycolic acid) [copolymer of 75:25 lactic acid:glycolic acid] 100 kDa (75/25 PLGA 100 kDa); 55% Dimethylsulfoxide (DMSO): 1.222 g of ISL, 1.8357 g PLGA 75/25 100 kDa (75/25 PLGA 76-115 kDa, Sigma Aldrich, Product #: 719927, Lot #: STBD7629V) and 3.6459 g of DMSO (product ID:41639 lot BCBH5215V from Sigma Aldrich) were added to a 20 mL scintillation vial and placed on an Eppendorf mixer set to 50° C. and 700 RPM for 3 days.

Composition 2: (10% ISL; 15% poly(lactic-co-glycolic acid) [copolymer of 50:50 lactic acid:glycolic acid] 29 kDa (50/50 PLGA 29 kDa); 75% N-Methyl-2-pyrrolidone (NMP)): 0.497 g of ISL, 0.7556 g PLGA 50/50 29 kDa (50/50 PLGA 28.5 kDa, Sigma Aldrich, Product #: 719870, Lot #: STBD5914V) and 3.742 g of NMP (product ID:41639 lot BCBH5215V from Sigma Aldrich) were added to a 20 mL scintillation vial and placed on an Eppendorf mixer set to 50° C. and 700 RPM for 3 days.

TABLE 5
Composition of injectable depot compositions of islatravir for rat in vivo studies

| | Mass of 4′-ethynyl- | | | | |
|---|---|---|---|---|---|
| | 2-fluoro-2′- | | | | Mass of |
| | deoxyadenosine (g) | Polymer | Polymer (g) | Solvent | Solvent (g) |
| Composition 1 | 1.222 g | Poly(lactic-co- | 1.8357 g | DMSO | 3.6459 g |
| | (18 wt %) | glycolic acid) | (27 wt %) | | (55 wt %) |
| | | [copolymer of | | | |
| | | 75:25 lactic | | | |
| | | acid:glycolic | | | |
| | | acid] 100 kDa | | | |
| Composition 2 | 0.497 g | poly (lactic-co- | 0.7556 g | NMP | 3.742 g |
| | (10 wt %) | glycolic acid) | (15 wt %) | | (75 wt %) |
| | | [copolymer of | | | |
| | | 50:50 lactic | | | |
| | | acid:glycolic | | | |
| | | acid] 29 kDa; | | | |
| | | 75% N-Methyl-2- | | | |
| | | pyrrolidone) | | | |

Drug input rates (i.e. drug release over time) were generated by deconvolution analysis. All deconvolution analyses were performed by employing the deconvolution module in the Phoenix WinNonlin 6.3 software (Pharsight, Certara Company). A unit impulse response (UIR) function was first established using intravenous bolus pharmacokinetic (PK) data from rats (data not shown). Next, mean implant PK profiles were deconvolved using these UIR parameters to yield absorption-time profiles, including both input rate and cumulative percent release.

TABLE 6
Islatravir rat in vivo plasma concentration
of islatravir injectable depot compositions.

| | 18% ISL; 27% poly(lactic-co- | | 10% ISL; 15% poly(lactic-co- | |
|---|---|---|---|---|
| | glycolic acid) [copolymer of | | glycolic acid) [copolymer of | |
| | 75:25 lactic acid:glycolic | | 50:50 lactic acid:glycolic | |
| | acid] 100 kDa; 55% | | acid] 29 kDa; 75% N-Methyl- | |
| | Dimethylsulfoxide | | 2-pyrrolidone | |
| | Islatravir plasma | | Islatravir plasma | |
| Time | concentration | Std. Dev. | concentration | Std. Dev. |
| (days) | (ng/ml) | (ng/mL) | (ng/ml) | (ng/mL) |
| 0.04 | 6 | 1 | 6 | 2 |
| 0.08 | 2.3 | 0.5 | 1.9 | 0.5 |
| 0.17 | 0.5 | 0.2 | 0.4 | 0.2 |
| 0.29 | 0.14 | 0.04 | 0.16 | 0.07 |
| 1 | 0.0393 | 0.0138 | 0.0432 | 0.0096 |
| 2 | 0.0249 | 0.0084 | 0.0306 | 0.0066 |
| 3 | 0.0192 | 0.0069 | 0.0246 | 0.0054 |
| 4 | 0.0171 | 0.0057 | 0.0192 | 0.0033 |
| 7 | 0.0123 | 0.0045 | 0.0168 | 0.0036 |
| 10 | 0.0096 | 0.0033 | 0.0129 | 0.0015 |
| 14 | 0.0081 | 0.0024 | 0.0141 | 0.0045 |
| 17 | 0.0069 | 0.0018 | 0.0126 | 0.0069 |
| 21 | 0.0054 | 0.0009 | | |
| 24 | 0.0051 | 0.0012 | | |
| 28 | 0.0042 | 0.0003 | | |
| 31 | 0.0036 | 0.0006 | | |
| 35 | 0.0033 | 0.0009 | | |
| 39 | 0.0033 | 0.0015 | | |
| 44 | 0.0036 | 0.0000 | | |
| 51 | 0.0033 | 0.0003 | | |
| 58 | 0.0024 | 0.0003 | | |
| 65 | 0.0024 | 0.0006 | | |
| 72 | 0.0018 | 0.0009 | | |
| 79 | 0.0027 | 0.0012 | | |
| 86 | 0.0018 | 0.0006 | | |
| 93 | 0.0018 | 0.0009 | | |
| 100 | 0.0009 | 0.0003 | | |
| 107 | 0.0006 | 0.0000 | | |
| 114 | 0.0006 | 0.0000 | | |

TABLE 7
Islatravir in vivo input rates from % Islatravir injectable
depot composition (18% ISL; 27% poly(lactic-co-glycolic
acid) [copolymer of 75:25 lactic acid:glycolic acid]
100 kDa; 55% Dimethylsulfoxide)

| Time | Input Rate |
|---|---|
| (Days) | (mg/day) |
| 1 | 0.31 |
| 5 | 0.12 |
| 10 | 0.08 |
| 15 | 0.06 |
| 20 | 0.05 |
| 25 | 0.04 |
| 30 | 0.03 |
| 40 | 0.03 |
| 50 | 0.03 |
| 60 | 0.02 |
| 70 | 0.02 |

### EXAMPLE 4

### In Vivo Release of Islatravir From Compositions With Variable Polymer and Polymer Loading

The preparation of the formulations for this example follows the methodology described in Example 1, and the in vitro drug release methods follow the description from Example 2. This example investigates influence of solvent type on in vitro release of islatravir from injectable depot compositions.

All animal studies were conducted following protocols in accordance with the Institutional Animal Care and Use Committee (IACUC) at NIRC and Merck & Co., Inc., Kenilworth, New Jersey, U.S.A. which adhere to the regulations outlined in the USDA Animal Welfare Act. For each composition tested, Wistar Han rats (N=6) were anesthetized using isoflurane prior to subcutaneous dose administrations. Each rat received islatravir injectable depot composition consisting of 50 mg/kg islatravir in various ratios and mixtures of poly(lactic-co-glycolic acid) [copolymer of 50:50 lactic acid:glycolic acid], N-Methyl-2-pyrrolidone [NMP], benzyl benzoate, and dimethyl sulfoxide [DMSO]), or vehicle only, as a single subcutaneous dose in the subscapular region at a dose volume of 0.3 or 0.5 mL/kg, as shown in the study design Table below. Six animals (3 males and 3 females) were used for each formulation. Animals were monitored until recovered. At indicated time points, samples of blood were obtained from anesthetized animals (using isoflurane) and processed to plasma for determination of islatravir levels.

Input rates were generated by deconvolution analysis. All deconvolution analyses were performed by employing the deconvolution module in the Phoenix WinNonlin 6.3 software (Pharsight, Certara Company). A unit impulse response (UIR) function was first established using intravenous bolus pharmacokinetic (PK) data from rats and macaques (data not shown). Next, mean implant PK profiles were deconvolved using these UIR parameters to yield absorption-time profiles, including both input rate and cumulative percent release.

TABLE 8
Islatravir (ISL) injectable depot composition in vivo study design

| | Injectable depot | Dose Level | Dose Volume |
|---|---|---|---|
| Group # | Composition (weight percent) | (mg/kg) | (mL/kg) |
| 50/50 PLGA in NMP | 14% ISL; 22% poly(lactic-co-glycolic | 50 | 0.5 |
| | acid) [copolymer of 50:50 lactic | | |
| | acid:glycolic acid] 29 kDa; 64% N- | | |
| | Methyl-2-pyrrolidone | | |
| 50/50 PLGA in BB/DMSO | 10% ISL; 15% poly(lactic-co-glycolic | 50 | 0.5 |
| | acid) [copolymer of 50:50 lactic | | |
| | acid:glycolic acid] 29 kDa; 54% Benzyl | | |
| | benzoate; 21% Dimethylsulfoxide | | |
| 50/50 PLGA in DMSO | 18% ISL; 27% poly(lactic-co-glycolic | 50 | 0.3 |
| | acid) [copolymer of 50:50 lactic | | |
| | acid:glycolic acid] 29 kDa; 55% | | |
| | Dimethylsulfoxide | | |

TABLE 9
Plasma concentration from islatravir injectable
depot compositions administered in rat.

| | 50/50 PLGA | | 50/50 PLGA | | 50/50 PLGA | |
|---|---|---|---|---|---|---|
| | in NMP | | in BB/DMSO | | in DMSO | |
| | | Std. | | Std. | | Std. |
| Time | Avg | Dev. | Avg | Dev. | Avg | Dev. |
| (days) | (ng/mL) | (ng/mL) | (ng/mL) | (ng/mL) | (ng/mL) | (ng/mL) |
| 0.04 | 9 | 1 | 3.0 | 0.7 | 6 | 2 |
| 0.08 | 5 | 2 | 1.2 | 0.3 | 1.9 | 0.5 |
| 0.2 | 1.0 | 0.6 | 0.5 | 0.2 | 0.4 | 0.1 |
| 0.3 | 0.13 | 0.08 | 0.23 | 0.07 | 0.16 | 0.06 |
| 1 | 0.0088 | 0.0003 | 0.13 | 0.04 | 0.043 | 0.009 |
| 2 | 0.004 | 0.001 | 0.10 | 0.04 | 0.030 | 0.005 |
| 3 | 0.026 | 0 | 0.07 | 0.03 | 0.024 | 0.004 |
| 4 | <LLQ | <LLQ | 0.03 | 0.01 | 0.019 | 0.003 |
| 7 | <LLQ | <LLQ | 0.010 | 0.009 | 0.016 | 0.004 |

Note:
both compositions were dosed at 50 mg/kg.

TABLE 10
Percent islatravir released from islatravir injectable
depot compositions administered in rat.

| | | |
|---|---|---|
| 50/50 PLGA | 50/50 PLGA | 50/50 PLGA |
| in NMP | in BB/DMSO | in DMSO |

| Time | Avg | Std. | Avg | Std. | Avg | Std. |
|---|---|---|---|---|---|---|
| (days) | (%) | Dev. | (%) | Dev. | (%) | Dev. |
| 0 | 0 | 0 | 0 | 0 | 0 | 0 |
| 0.2 | 16 | 4 | 9 | 0 | 16 | 0 |
| 1 | 64 | 11 | 25 | 1 | 25 | 0 |
| 2 | 78 | 8 | 36 | 3 | 25 | 0 |
| 4 | 87 | 4 | 58 | 7 | 26 | 1 |
| 7 | 91 | 1 | 85 | 7 | 25 | 0 |
| 20 | 90 | 1 | 93 | 0 | 79 | 0 |

Note:
both compositions were dosed at 50 mg/kg.

1. An injectable depot composition comprising 10% to 50% of a biocompatible bioerodible polymer by weight, 3% to 40% of islatravir, or a pharmaceutically acceptable salt thereof, by weight and 30% to 85% of a solvent by weight.

2. The injectable depot composition of claim 1 wherein the biocompatible bioerodible polymer is selected from the group consisting of poly(DL-lactide), poly(caprolactone), poly(lactic-co-glycolic acid), poly(lactide), poly(glycolide), poly(ε-caprolactone), poly(ortho esters), poly(dioxanone) and combinations thereof.

3. The implant drug delivery system of claim 2 wherein the biocompatible bioerodible polymer is poly(lactic-co-glycolic acid).

4. The injectable depot composition of claim 1 wherein the solvent is selected from the group consisting of ethyl benzoate, N-methyl-2-Pyrrolidone, dimethylsulfoxide, benzyl benzoate, benzyl alcohol, poly(ethylene glycol) dimethyl ether, triacetin, glycofurol and mixtures thereof.

5. The injectable depot composition of claim 4 wherein the solvent is dimethylsulfoxide.

6. The injectable depot composition of claim 1 wherein the islatravir, or a pharmaceutically acceptable salt thereof, is present in the composition between 8% to 20% by weight.

7. The injectable depot composition of claim 1 wherein the biocompatible bioerodible polymer is present in the composition between 15% to 30% by weight.

8. The injectable depot composition of claim 1 wherein the solvent is present in the composition between 55% to 75% by weight.

9. The injectable depot composition of claim 1 wherein the islatravir, or a pharmaceutically acceptable salt thereof, is present in the composition between 8% to 18% by weight, the polymer is present in the composition between 15% to 27% by weight and the solvent is present in the composition between 55% to 75% by weight.

10. The injectable depot composition of claim 9 wherein the biocompatible bioerodible polymer is poly(lactic-co-glycolic acid).

11. The injectable depot composition of claim 10 wherein the ratio of the lactic acid to glycolic acid of the poly(lactic-co-glycolic acid) is 75:25.

12. The injectable depot composition of claim 11 wherein the weight of the 75:25 poly(lactic-co-glycolic acid) is 100 kDa.

13. The injectable depot composition of claim 10 wherein the ratio of the lactic acid to glycolic acid of the poly(lactic-co-glycolic acid) is 50:50.

14. The injectable depot composition of claim 13 wherein the weight of the 50:50 poly(lactic-co-glycolic acid) is 29 kDa.

15. The injectable depot composition of claim 9 wherein the solvent is dimethylsulfoxide.

16. The injectable depot composition of claim 1 which comprises 18% of islatravir by weight, 27% of 75:25 poly(lactic-co-glycolic acid) 100 kDa by weight and 55% dimethylsulfoxide by weight.

17. The injectable depot composition of claim 1 which comprises 18% of islatravir by weight, 27% of 50:50 poly(lactic-co-glycolic acid) 29 kDa by weight and 55% dimethylsulfoxide by weight.

18. The injectable depot composition of claim 1 which forms a biodegradable implant in situ.

19. The injectable depot composition of claim 1 which is injected into a patient in need thereof subdermally.

20. The injectable depot composition of claim 1 wherein the islatravir is released at therapeutic concentrations for one month.

21. The injectable depot composition of claim 1 wherein the islatravir is released at therapeutic concentrations between three and six months.

22. A method of treating HIV infection by administering to a subject by injection the composition of claim 1.

23. A method of preventing HIV infection by administering to a subject by injection the composition of claim 1.
